(19) United States
(12) Patent Application Publication
(10) Pub. No.: US 2014/0303695 A1
(43) Pub. Date: Oct. 9, 2014

(54) APPARATUSES FOR PROVIDING THERMAL TREATMENT AND METHODS FOR USING THE APPARATUSES

(71) Applicant: Paul A. Komorita, Houston, TX (US)

(72) Inventor: Paul A. Komorita, Houston, TX (US)

(21) Appl. No.: 13/858,460

(22) Filed: Apr. 8, 2013

(51) Int. Cl. A61F 7/08; A61F 7/10

(52) U.S. Cl. CPC A61F 7/08; A61F 7/103
USPC 607/104

(57) ABSTRACT

Apparatuses and methods for providing thermal treatment to a foot and ankle of a wearer are provided. An example apparatus may allow applying cold or heat to the foot and ankle of the wearer through a liquid contained in one or more hollows in a top portion or a sole portion of the apparatus. The top portion may have a form of a boot and include a fluid-tight material. The top portion may be filled with the liquid using a top portion filling member disposed on an upper part of the top portion, then the top portion filling member may be sealed using a top portion sealing member. The sole portion may be attached to the top portion removably or non-removably. The sole portion may include a hollow, which may contain the liquid or be filled and emptied using a sole portion filling member and a sole portion sealing member.

### CROSS REFERENCE TO RELATED APPLICATION

This Application claims priority to U.S. Provisional Utility Application Ser. No. 61/740,785 filed Dec. 21, 2012, the contents of which is incorporated by this reference.

### TECHNICAL FIELD

This disclosure generally relates to thermal treatment and apparatuses for thermal treatment of various body parts and regions and, more specifically, to apparatuses for providing thermal treatment to a foot and ankle of a wearer and methods for using the apparatuses.

### BACKGROUND

Cold and heat are widely used in medicine and sports to treat acute or chronic conditions and injuries. Temperature alterations produce various effects on body tissues, including relieving pain, relaxing muscles, reducing swelling and inflammation, increasing tissue elasticity, and producing other effects. Depending on the purpose of applying cold or heat, duration of such treatment may vary.

Conventional devices used to provide cold or heat treatment include hot water bottles, electric blankets, cold packs and wraps, and cold and hot bags. Hot water bottles and cold and hot bags may be used to provide cold and hot treatments, but they may not be comfortable to use for long periods, or when walking or moving. Cold packs and wraps are fixed on a human body and can be used when a wearer walks or moves, but provide only cold treatment. In addition to that, hot water bottles as well as cold packs and wraps are usually applied to only a limited area of a human body. Electric blankets are connected to the electric network by a cable, so user movements are limited.

### BRIEF DESCRIPTION OF THE DISCLOSURE

According to one or more example embodiments of the disclosure, there are provided apparatuses and methods for providing thermal treatment that can be applied to various body parts and regions, however, by way of example and without limitation, the embodiments of the disclosure relate to the thermal treatment of a foot and an ankle of a wearer using preliminary heated or cooled liquid added in the apparatus. The apparatus, having a form of a boot, may be put on a foot of a wearer to produce effects of a hot water bottle on a whole surface of the foot and ankle. The apparatus may be used when the wearer moves or walks. Thus, the wearer may have the freedom of movements when using the apparatus.

According to various example embodiments, there is provided an apparatus for providing thermal treatment to a foot and an ankle of a wearer comprising a sole portion, and a top portion having a form of a boot and attached to the sole portion. The top portion may include a fluid-tight material and a hollow that is configurable to be filled with a liquid. Additionally, the apparatus may comprise a top portion filling member attached to the top portion in a fluid-tight manner. The top portion filling member may connect with the hollow, thus allowing filling the hollow with the liquid and/or emptying the hollow. The top portion filling member may be sealed with a top portion sealing member in the fluid-tight manner.

The sole portion may be permanently attached to the top portion. Alternatively, the sole portion may be removeably attached to the top portion using one or more of a clasp, Velcro, a zip fastening, a fastening belt, and so forth. The sole portion may also include the fluid-tight material and a hollow sole portion that is configurable to be filled with the liquid. For adding the liquid to the sole portion, the sole portion may have a sole portion filling member and a sole portion sealing member.

Additional systems, methods, apparatuses, features, and aspects are realized through the techniques of various embodiments of the disclosure. Other embodiments and aspects of the disclosure are described in detail below and are considered a part of the claimed disclosure. Other embodiments and aspects can be understood with reference to the description and the drawings.

### BRIEF DESCRIPTION OF THE DRAWINGS

Having thus described the disclosure in general terms, reference will now be made to the accompanying drawings, which are not necessarily drawn to scale, and wherein:

FIG. 1 shows an exploded view of an example apparatus for providing thermal treatment to a foot and an ankle of a wearer, in accordance to some example embodiments.

FIG. 2 shows an example apparatus for providing thermal treatment to a foot and an ankle of a wearer, in accordance to some example embodiments.

FIG. 3 shows an example apparatus for providing thermal treatment to a foot and an ankle of a wearer with non-removable attachment of a sole portion, in accordance to some example embodiments.

FIG. 4 shows an example apparatus for providing thermal treatment to a foot and an ankle of a wearer with a removable attachment of a sole portion, in accordance to some example embodiments.

FIG. 5A shows an example apparatus for providing thermal treatment to a foot and an ankle of a wearer with a sole portion removably attached using band fasteners, in accordance to some example embodiments.

FIG. 5B shows an example apparatus for providing thermal treatment to a foot and an ankle of a wearer configured for removable attachment of a sole portion using band fasteners, in accordance to some example embodiments.

FIG. 6 shows an example apparatus for providing thermal treatment to a foot and an ankle of a wearer comprising one or more fasteners, in accordance to some example embodiments.

FIG. 7 shows an example apparatus for providing thermal treatment with ice cubes placed in the apparatus, in accordance to some example embodiments.

FIG. 8 shows an alternative embodiment of an apparatus for providing thermal treatment, in accordance to some example embodiments.

FIG. 9 shows an alternative embodiment of an apparatus for providing thermal treatment, in accordance to some example embodiments.

FIG. 10 shows an example flow diagram of a method for using an apparatus for providing thermal treatment to a foot and an ankle of a wearer, according to one or more embodiments.

### DETAILED DESCRIPTION

Illustrative embodiments of the disclosure now will be described more fully hereinafter with reference to the accompanying drawings, in which some but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. Like numbers refer to like elements throughout.

Overview

Apparatuses and methods disclosed herein enable providing thermal treatment to various body parts and regions, but more specifically, to a foot and an ankle of a wearer without restricting movements of a wearer. And wherein the treatment is intended to, at least in most embodiments, encapsulate the entire foot and ankle. An apparatus for providing thermal treatment to a foot of a wearer may be filled with hot or cold liquid (e.g. water) to apply cold or heat to the foot and/or ankle of the wearer. The apparatus may have a form of a boot with one or more hollows in side and/or sole parts. Before filling the apparatus with the liquid, the apparatus may be put on the foot of the wearer. Then, heated or chilled liquid may be poured in the hollows through a filling member disposed on the upper part of the apparatus. Due to the liquid spreading in the hollows, the apparatus may embrace the foot of the wearer, thus ensuring adherence to the foot and comfort of the wearer. To additionally fix the apparatus around the foot and ankle, the apparatus may comprise a clasp, Velcro, a zip fastening, a tie-wrap, a fastening belt, and so forth. After the liquid is poured, the filling member may be sealed with a sealing member.

When the wearer stands or walks with the apparatuses on his feet, the liquid that may be in the sole part of the apparatus may move upwards under the weight of the wearer. In this case, heat or cold is not applied to the soles of the feet of the wearer. To avoid that, in some embodiments, the apparatus may comprise a top portion, which may be filled with the liquid, and a sole portion, which may or may not be filled with the liquid. The top portion and the sole portion may be separated from each other preventing the liquid from flowing between them.

In some embodiments, when the sole portion may be filled with the liquid and emptied after use, the sole portion may comprise a filling member and a sealing member.

To avoid perforation or damage when walking or standing, the sole portion, if any, or the sole part of the apparatus contacting a floor or ground may be reinforced or include durable materials protecting the top portion. For example, the surface contacting a floor or ground may have an additional durable covering and/or layer. In some embodiments, the sole portion itself may play a role of such a covering or layer.

The sole portion may be permanently or removeably attached to the top portion. Removable attachment may be performed using a clasp, Velcro, a zip fastening, a tie-wrap, a fastening belt, and/or other means.

Additionally, to provide extra heat or cold and/or comfort to a foot of the wearer, the apparatus may comprise one or more insoles that may be inserted in the apparatus and removed from it. The insole may be filled with water, a water solution, a polymer gel, an oil, a wax, and so forth. The insole may be cooled, frozen, or heated. Cooling or heating may be performed using a cooling or heating device (for example, a refrigerator, a microwave oven, and so forth), cold or hot water, or otherwise.

In some embodiments, the apparatus may comprise a set of insoles having different shapes, made of different materials, and/or filled with different liquids.

In an alternative embodiment, the apparatus may have no inner wall separating a skin of the wearer from the liquid. Thus, the liquid may be poured inside the top portion forming the cavity where the foot of the wearer is placed. The apparatus may be secured on the foot and ankle of the wearer using a gasket preventing the liquid from escaping. The gasket may be secured by a clasp, Velcro, a zip fastening, fastening belts, and so forth.

Due to direct contact with the skin of the wearer in the latter embodiment, various substances and/or medications may be added in the liquid used in the apparatus. For example, the liquid in the latter embodiment may include water, a water solution, a decoction, an infusion, an extract, an oil, and so forth.

Example Embodiments of the Apparatus

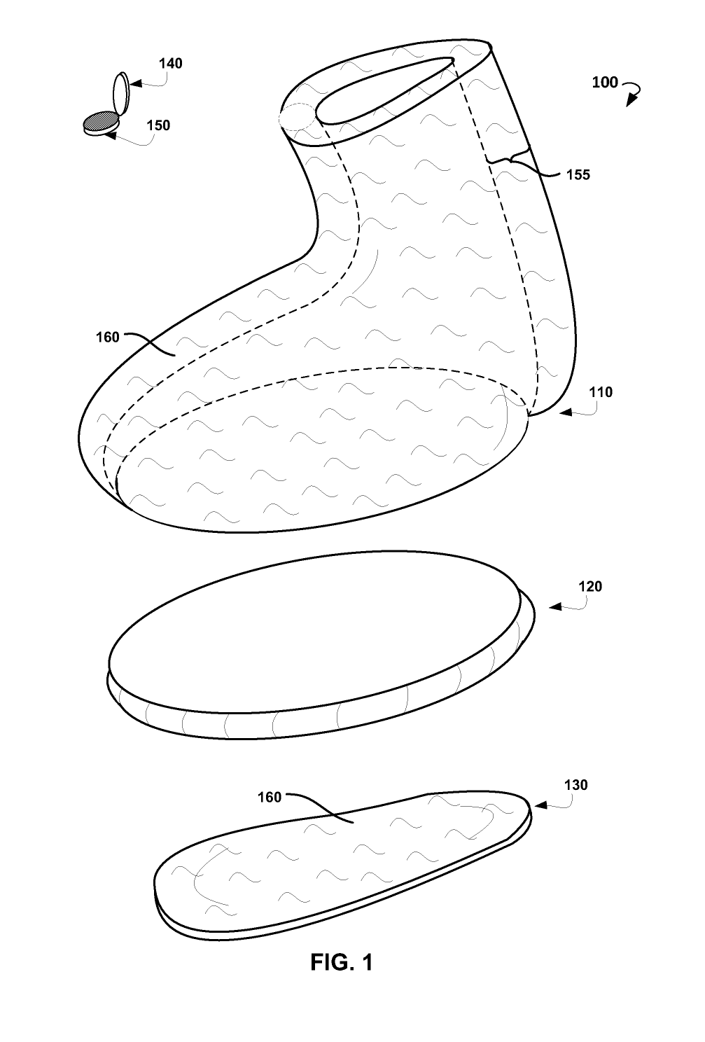

Referring now to the drawings, FIG. 1 shows an exploded view of an example apparatus 100 for providing thermal treatment to the entire surface area of the foot and an ankle of a wearer, in accordance to some example embodiments. As shown in FIG. 1, the apparatus 100 may include a top portion 110, a sole portion 120, and a filling member 150 that may be sealed with a sealing member 140. The top portion 110 may have a form of a human foot and may have a hollow 155 throughout that may be filled with a liquid 160. The top portion 110 may have the filling member 150 disposed in an upper part of the top portion 110.

The top portion 110 may include a fluid-tight material (for example, natural rubber, flexible plastic, polyvinyl chloride (PVC), silicon, and so forth). The fluid tight material may allow the top portion 110 to be filled with the liquid 160 and prevent leaking of the liquid 160. The liquid 160, which may be poured in the top portion 110, may include water, a water solution, a polymer gel, an oil, a wax, and so forth.

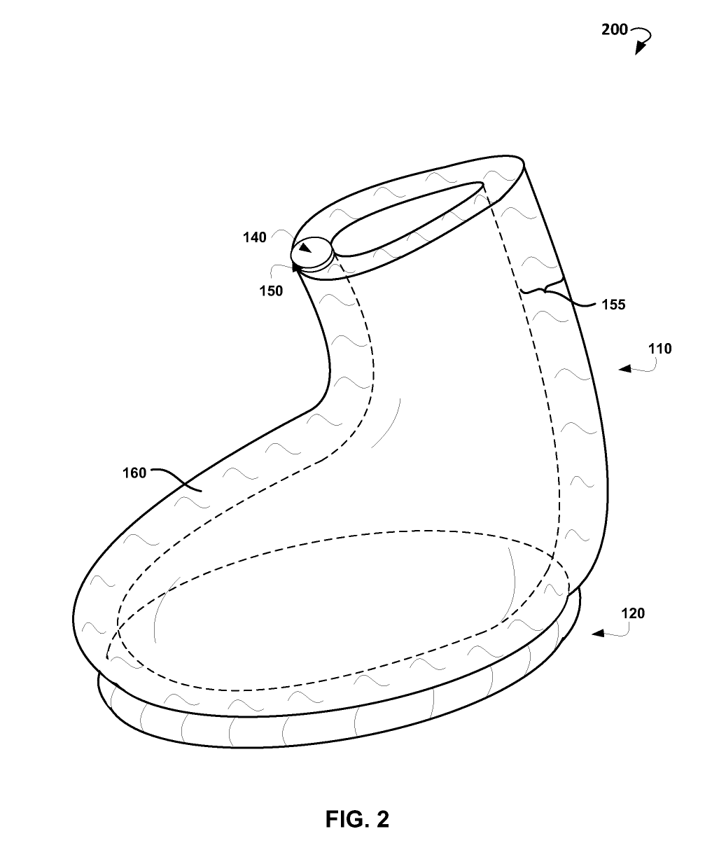

The top portion 110 may be attached to the sole portion 120 in a removable or non-removable manner. In various embodiments, the sole portion 120 may be filled with the hot or cold liquid 160 and emptied after usage, permanently filled with the liquid 160, or serve as a protection for the top portion 110 to keep it from damage and perforation. Various embodiments of the sole portion 120 attachment and implementation will be described below with reference to FIGS. 2-5.

Still referring to FIG. 1, the top portion 110 may include the filling member 150 to fill the top portion 110 with the liquid 160. The filling member 150 may be disposed in the upper part of the top portion 110 to facilitate adding the liquid 160 when the apparatus 100 is on the foot of the wearer and avoid spillage. The filling member 150 may be attached to the top portion 110 in a fluid-tight manner and may communicate with the hollow 155 so that the liquid 160 poured in the filling member 150 is received by the hollow 155. The filling member 150 may be sealed with the sealing member 140 to keep the liquid 160 inside the hollow 155.

In some embodiments, the apparatus 100 may comprise at least one removable insole 130. The insole may be inserted in the top portion 110 to provide additional thermal treatment and comfort to the foot of the wearer. The insole 130 may include a fluid-tight material and/or other materials. The fluid-tight material may provide for filling the insole 130 with the liquid 160 so that the insole 130 could accumulate heat or cold when heated, cooled, or frozen. Heating or cooling may be done using a cooling or heating device (for example, a microwave oven, a freezer, and so forth). Other materials may ensure the comfort of the foot of the wearer.

Additionally, the apparatus 100 may comprise at least one removable insert (not shown). One or more inserts may be positioned inside the apparatus 100 so that the inserts were applied to the area of a foot or ankle requiring hot or cold treatment. The inserts may include a fluid-tight material and be filled with the liquid 160. The inserts may be heated, cooled, or frozen using a cooling or heating device.

In some embodiments, the apparatus 100 may comprise fixing means to fix one or more inserts in a desired position. Additionally, some inserts may be supported by other inserts that may be in a position in the apparatus. For example, inserts configured to be applied to the left and right foot areas may be individually removed and reinserted since they do not require support. However, the inserts configured to be applied to the left and right ankle areas may be supported by the inserts for foot, and thus may be inserted together with only the inserts for foot.

In some embodiments, the apparatus 100 may comprise a set of removable insoles and/or inserts designed for various purposes and having different properties due to different liquids used to fill them and different materials from which they are made.

FIG. 2 shows an example apparatus 200 for providing thermal treatment to a foot and an ankle of a wearer, in accordance to some example embodiments. The apparatus 200 includes a top portion 110 and a sole portion 120 attached to each other in a removable or non-removable manner. As shown by FIG. 2, the top portion 110 has a hollow 155 adjacent to the sides and a sole of the foot and to the ankle of the wearer. Thus, when the top portion 110 is filled with a liquid 160, the liquid 160 spreads around the foot and ankle, and thermal treatment may be applied both to the sides and a sole of the foot and to the ankle of the wearer.

The sole portion 120 may include durable materials to protect the top portion 110 from punctures, piercing, and other damage. The sole portion 120 may be filled with the liquid 160 and provide for cooling or heating of the foot and ankle of the wearer or may perform a protective function only. The liquid 160 may include water, a water solution, a polymer gel, an oil, a wax, and so forth.

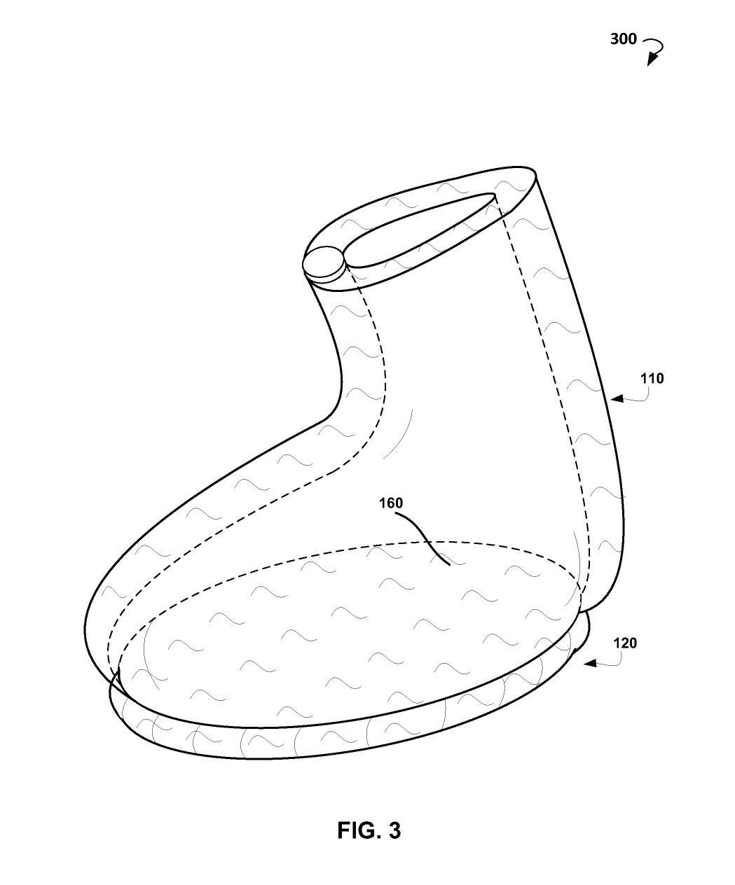

FIG. 3 shows an example apparatus 300 for providing thermal treatment to a foot and an ankle of a wearer with non-removable attachment of a sole portion, in accordance to some example embodiments. The apparatus 300 includes a top portion 110 and a sole portion 120 that are permanently attached to each other. The sole portion 120 may be filled with a liquid 160 to apply heat or cold to a sole of the foot of the wearer. The liquid 160 may include water, a water solution, a polymer gel, an oil, a wax, and so forth.

In some embodiments, the sole portion 120 may include a filling member and a sealing member (not shown) to fill, refill, and empty the sole portion 120. In other embodiments, the sole portion 120 may have no filling capability. The liquid 160 may constantly stay in the hollow in the sole portion 120. In that case, the apparatus 300 may be placed in a cooling or heating device so that the liquid 160 accumulates heat or cold.

In other embodiments, the sole portion 120 may be removably attached to the top portion 110.

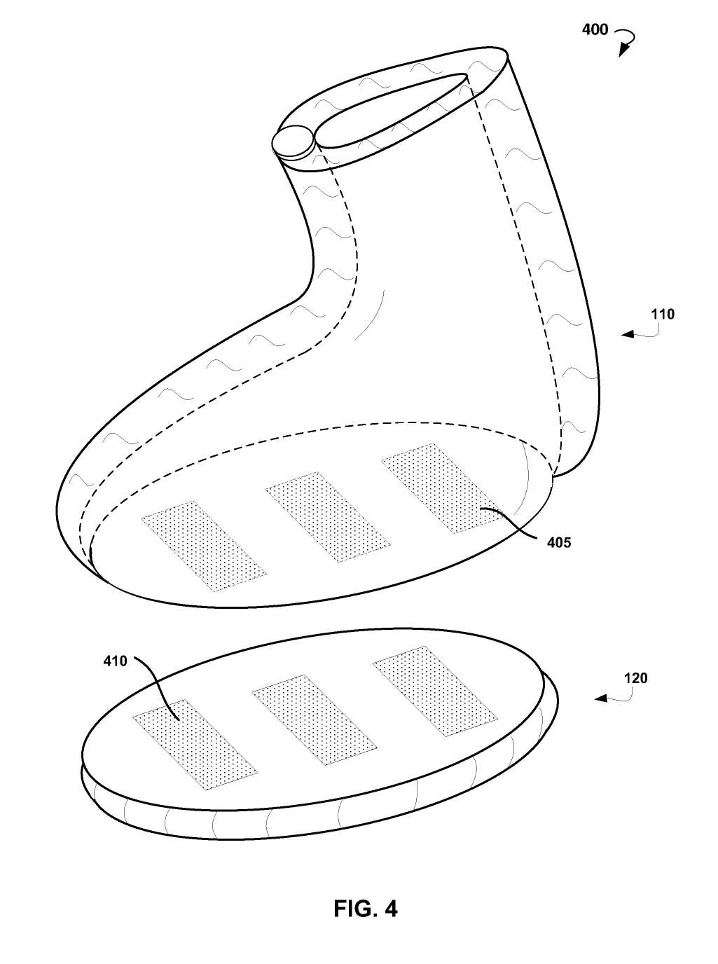

FIG. 4 shows an example apparatus 400 for providing thermal treatment to a foot and an ankle of a wearer with a removable attachment of a sole portion, in accordance to some example embodiments. The top portion 110 may be removably attached to the sole portion 120 using Velcro. As shown by FIG. 4, matching pieces of Velcro may be attached to the top portion 110 and the sole portion 120. For example, loop side pieces 405 of Velcro may be attached to the top portion 110, and hook side pieces 410 of Velcro may be attached to the sole portion 120 or vice versa.

In some example embodiments, attachment using Velcro may be combined with one or more of other types of fastening (e.g. a clasp, a zip fastening, a band fastener, a tie-wrap, a fastening belt, and so forth).

In the embodiment shown by FIG. 4, the sole portion 120 may be filled or not filled with a liquid. The sole portion 120 may be filled with the liquid before using the apparatus 400 and emptied afterwards using a filling member (not shown). Alternatively, the sole portion 120 may be permanently filled with the liquid without a capability of emptying it. When this is the case, the sole portion 120 may be heated, cooled, or frozen in a cooling or heating device.

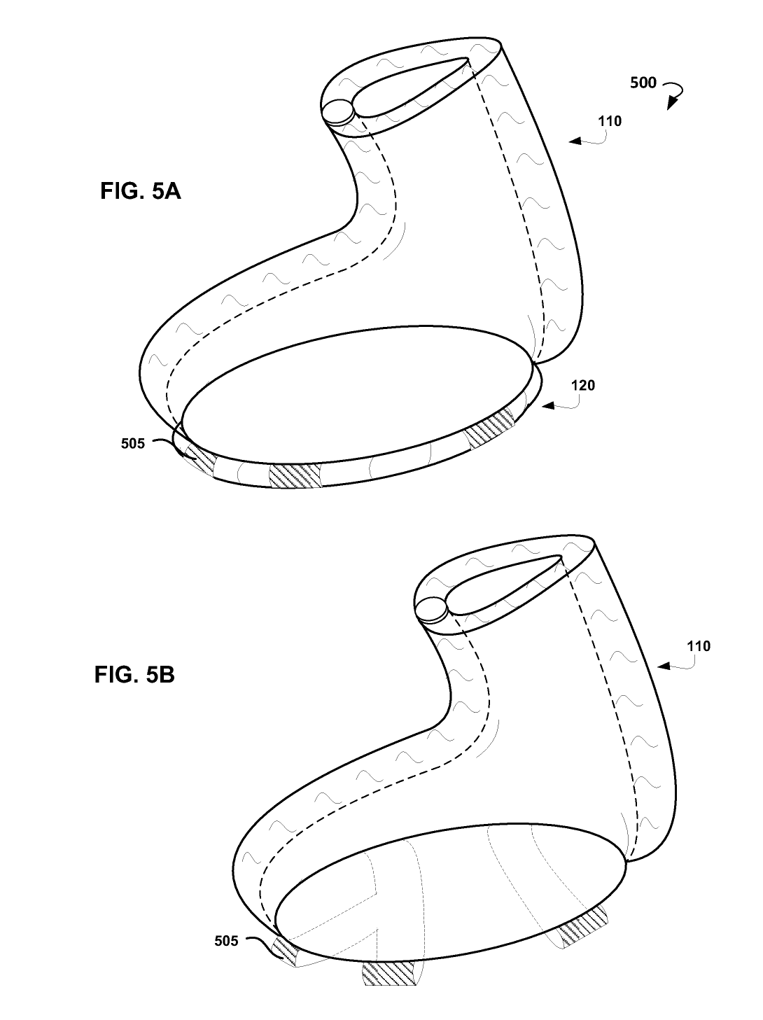

According to other embodiments, the sole portion 120 may be removably attached to the top portion 110 using other types of fastening, for example, using one or more band fasteners, as illustrated by FIGS. 5A and 5B.

FIG. 5A shows an example apparatus 500 for providing thermal treatment to a foot and an ankle of a wearer with a sole portion removably attached using band fasteners, in accordance to some example embodiments. The top portion 110 may be removably attached to the sole portion 120 using band fasteners 505. The sole portion may be inserted in the band fasteners 505 and removed when necessary.

The band fasteners 505 may include a band attached to the top portion 110 so that the sole portion 120 is tightly fixed when inserted in a loop formed by the band.

FIG. 5B shows an example apparatus 500 for providing thermal treatment to a foot and an ankle of a wearer configured for removable attachment of a sole portion using band fasteners, in accordance to some example embodiments. As shown by FIG. 5B, one or more bands may be attached by one or more sides to the top portion 110 to form a loop. The loop may be configured to match the size of the sole portion 120 to tightly fix the sole portion 120.

To ensure secure fixing, band fasteners 505 may be combined with one or more other types of fastening (e.g. a clasp, Velcro, a zip fastening, a tie-wrap, a fastening belt, and so forth).

Moreover, according to some embodiments, the apparatus for providing thermal treatment to a foot and an ankle of a wearer may be secured around a foot and ankle of a wearer using securing means.

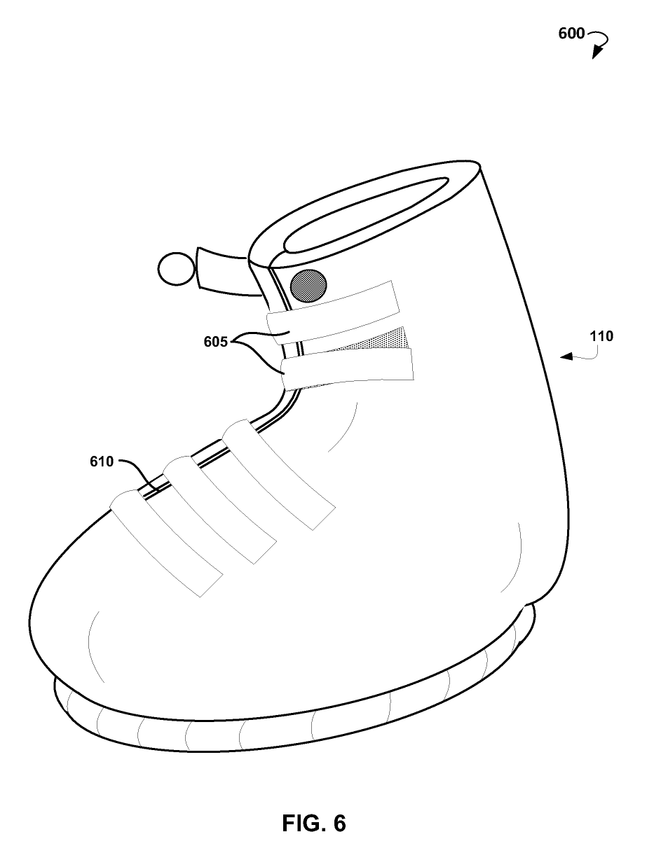

As shown by FIG. 6, an apparatus 600 for providing thermal treatment to a foot and an ankle of a wearer may comprise one or more fasteners, for example, Velcro fasteners 605, to secure the apparatus 600 around the foot and ankle. The Velcro fasteners 605 may be attached to the top portion 110 to prevent loose fit of the apparatus 600 on the foot of the wearer. Loose fit of the apparatus 600 may be uncomfortable for the wearer and result in the apparatus 600 falling off the foot of the wearer, and is therefore to be avoided. The Velcro fasteners 605 may ensure adjustable fastening of the apparatus 600 for its secure fixing.

To ensure comfortable placing of a foot in the apparatus 600, the top portion 110 may have a front opening 610. The front opening 610 may extend from the front upper part of the top portion 110 to the toes. Thus, the wearer may place a foot in the apparatus 600 without disturbing in case of an injured foot or ankle.

In some embodiments, the apparatus 600 may additionally comprise other fastening means to provide extra fixing of the apparatus 600 around the foot and ankle of the wearer. Other fastening means may include one or more clasps, zip fastenings, band fasteners, tie-wraps, fastening belts, and so forth.

According to various embodiments, the apparatus for providing thermal treatment to a foot and an ankle of a wearer may be filled with various liquids. To provide cold treatment, the apparatus may be filled with water in a liquid of frozen state.

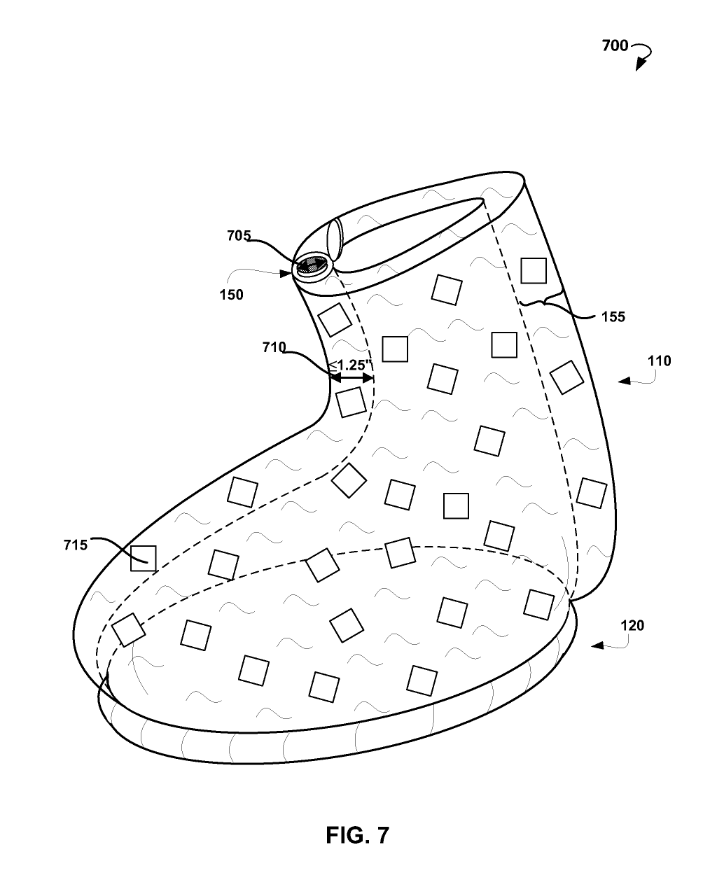

FIG. 7 shows an example apparatus 700 for providing thermal treatment with ice cubes placed in the apparatus, in accordance to some example embodiments. One or more ice cubes 715 may have side lengths of approximately 1.25 inches. The ice cubes 715 may be placed in the top portion 110 and/or the sole portion 120 through the filling member 150. The filling member 150 may have an opening 705 with an inner diameter of at least 1.25 inches to receive the ice cubes 715 having sides of 1.25 inches. The ice cubes 715 may then spread in the hollow 155 inside the top portion 110 and/or a hollow (if any) inside the sole portion 120.

In some embodiments, the sole portion 120 may not communicate with the top portion 110 and may comprise a sole portion filling member (not shown) configured to receive the ice cubes 715 having sides of 1.25 inches.

Additionally, the top portion 110 and the sole portion 120 may be configured to receive the ice cubes 715. For example, space 710 between sides of the top portion 110 forming the hollow 155 may be at least 1.25 inches.

Ice cubes 715 placed in the apparatus 700 may provide for prolonged cooling effect, thus allowing the wearer to receive cold treatment for a long period without refilling the apparatus 700.

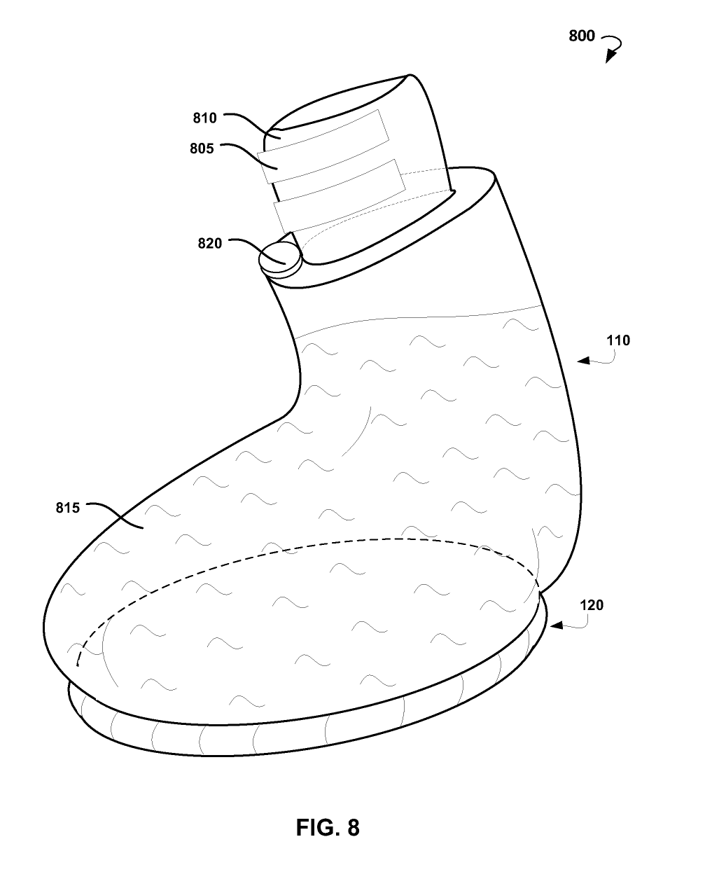

An alternative embodiment of the apparatus for providing thermal treatment is depicted with reference to FIG. 8.

According to FIG. 8, an apparatus 800 for providing thermal treatment may comprise a top portion 110, a sole portion 120, a filling member 820, a gasket 810, and one or more fasteners 805. The top portion 110 may include a fluid-tight material, for example, natural rubber, flexible plastic, PVC, silicon, and so forth. A liquid 815 may be poured in the top portion 110 through the gasket 810. Since the liquid 815 may directly contact skin of the foot and ankle of the wearer when the apparatus 800 is in use, various medications, extracts of plants or vegetables, decoctions, salts, tea, herbs, juices, oils, soap, and other substances may be added to the liquid 815. Thus, the liquid 815 may include one or more of water, a water solution, a decoction, an infusion, an extract, an oil, and so forth. Due to that, the apparatus 800 may be used as a foot bath that does not restrict movements of the wearer. The fluid-tight material may allow keeping the liquid 815 inside the top portion 110 without leaking of the liquid 815.

The top portion 110 may be put on the foot of the wearer, and the foot and ankle may directly contact the liquid 815 inside the top portion. To prevent escaping of the liquid 815, the top portion 110 may be secured around an ankle of the wearer using the gasket 810 fastened by the one or more fasteners 805. The gasket 810 may include foam rubber to ensure close contact with the skin of the wearer and keep the liquid 815 from spillage, for example, when the wearer moves or walks.

In some embodiments, the apparatus 800 may include the filling member 820 and a sealing member configured to seal the filling member 820. The filling member 820 may be used to fill the top portion 110 with the liquid 815 and/or refill the top portion 110.

In some embodiments, the top portion 110 may be removably or non-removably attached to the sole portion 120. In some embodiments, the sole portion 120 may have a hollow (not shown) that may be permanently filled with a liquid or may be filled and emptied after use. In other embodiments, the sole portion 120 may include durable material and serve to protect the top portion 110 from damage and perforation and/or perform other functions. Variants of attachment of the sole portion 120 to the top portion 110 are described above with reference to FIGS. 2-5.

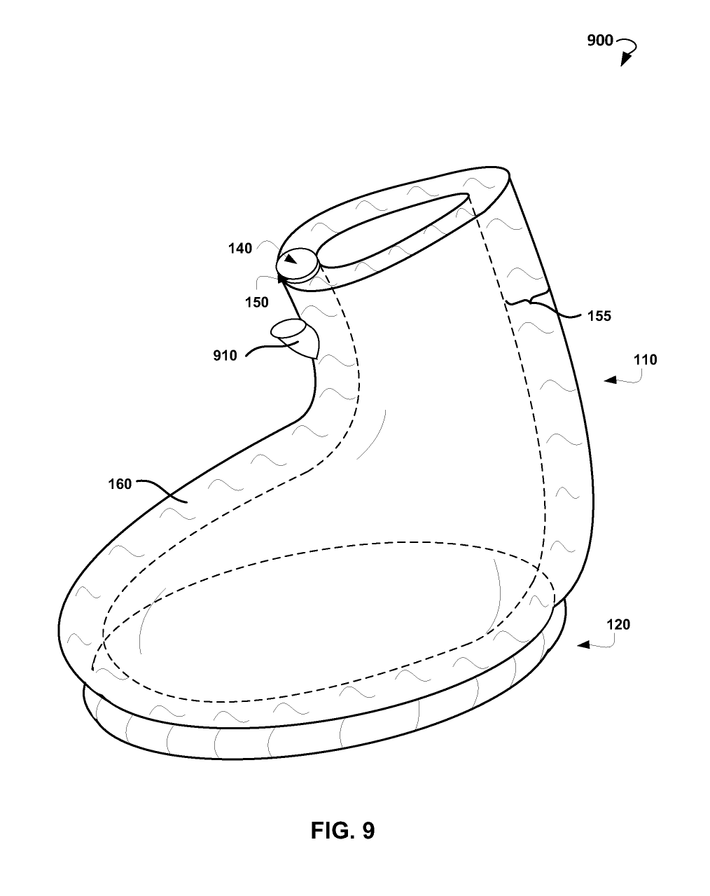

As shown by FIG. 9, an apparatus 900 for providing thermal treatment may a valve 910 attached to the top portion 110 in the fluid-tight manner, in accordance to some embodiments. The valve may communicate with the hollow 155 and provide for regulating and controlling the liquid 160 and/or gases (for example, air) by opening, closing, or partially opening or closing some passageways. The valve 910 may be disposed in a front position (as shown in FIG. 9) or any other position, for example, lateral or back position. In some embodiments, the valve 910 may be used to fill, refill, or empty the hollow 155 with the liquid 160.

The apparatus for providing thermal treatment may be used when a wearer moves or walks, thus not restricting the freedom of movements as conventional devices. Refilling capability allows the wearer to use the apparatus for a desired period without placing it to a cooling or heating device to accumulate cold or heat.

Example Operation Method

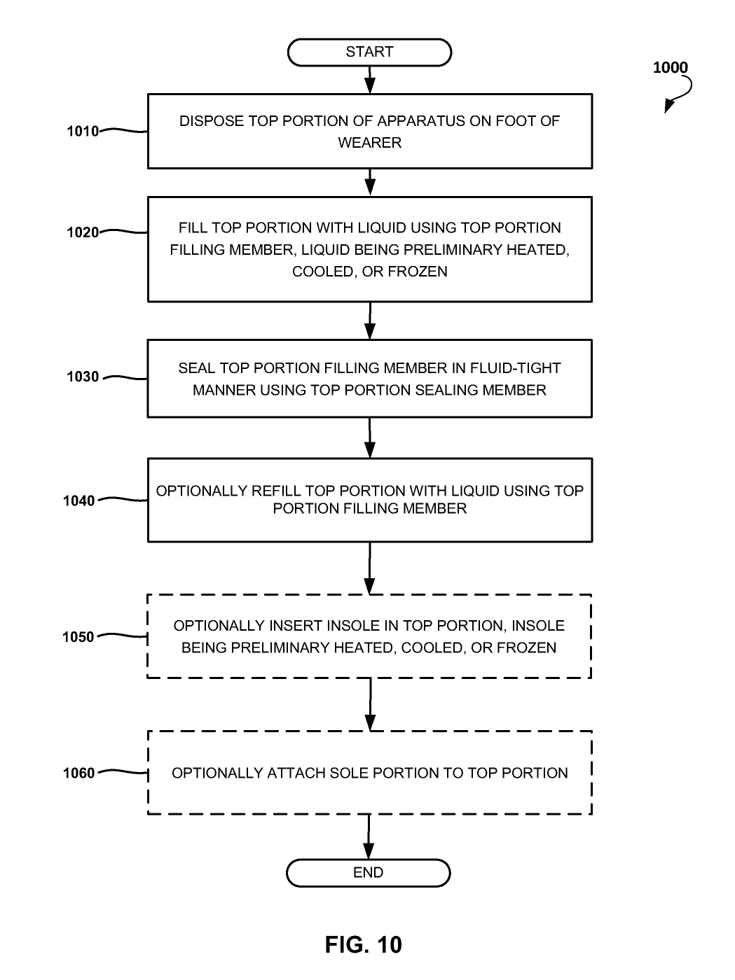

FIG. 10 shows an example flow diagram of a method 1000 for using an apparatus for providing thermal treatment to a foot and an ankle of a wearer, according to one or more embodiments of the disclosure. The method 1000 may be implemented by the apparatus as described above with reference to FIGS. 1-9.

The method 1000 may commence in operation 1010 with disposing a top portion 110 of the apparatus for providing thermal treatment on a foot and an ankle of the wearer. The apparatus may be secured on the foot and ankle of the wearer using one or more fasteners 605. The fasteners may include a clasp, Velcro, a zip fastening, a tie-wrap, a fastening belt, and so forth. In operation 1020, the top portion 110 may be filled with a liquid 160 using a top portion filling member 150. The liquid 160 may include water, a water solution, one or more ice cubes, a polymer gel, an oil, a wax, and so forth. To provide hot or cold treatment, the liquid 160 may be preliminary heated, cooled, or frozen to a desired temperature. The top portion filling member 150 may be then sealed in a fluid-tight manner using a top portion sealing member 140 in operation 1030. Since the top portion 110 includes a fluid-tight material, the liquid 160 may be prevented from leaking. Thus, the liquid 160 may be kept from spilling or leaking when the wearer of the apparatus moves or walks.

With the lapse of time, the temperature of the liquid 160 may tend to the surrounding temperature. In operation 1040, when the wearer wishes to continue thermal treatment, he may optionally refill the top portion 110 with the liquid 160 using the top portion filling member 150.

In some embodiments, the apparatus may comprise one or more insoles 130 configured to include the liquid 160. The one or more insoles 130 may be heated or cooled in a heating or cooling device, for example, a freezer, a microwave oven, and so forth. The insole, preliminarily heated, cooled, or frozen in such a way, may be optionally inserted in the top portion 110 of the apparatus in operation 1050 to ensure additional thermal effect and/or comfort to the foot of the wearer.

In some embodiments, the top portion 110 of the apparatus may be optionally attached to the sole portion 120 in operation 1060. Various ways of attaching the sole portion 120 and the top portion 110 are described above with reference to FIGS. 2-5. In brief, they may include a clasp, Velcro, a zip fastening, at least one fastening belt, and so forth.

Although the embodiments have been described with reference to specific example embodiments, it will be evident that various modifications and changes can be made to these example embodiments without departing from the broader spirit and scope of the present application. Further, it is contemplated by the Applicant that many of the benefits of treatment made available to the wearer because of the structures described above my easily apply for other parts or areas of the body. For example, structures similar in basic form to those as described above could be adapted for use in the hot or cold treatment of a wearer's arm, hand, wrist, shoulder, leg, knee, neck, head or any other body part or area. Accordingly, the specification and drawings are to be regarded in an illustrative rather than a restrictive sense.

What is claimed is:

1. An apparatus for providing thermal treatment to a foot and an ankle of a wearer, the apparatus comprising:
a sole portion;
a top portion having a form of a boot and attached to the sole portion, the top portion including a fluid-tight material and a hollow configurable to be filled with a liquid;
at least one top portion filling member attached to the top portion in a fluid-tight manner and connecting with the hollow, the at least one top portion filling member being configurable to fill and/or empty the hollow with the liquid; and
at least one top portion sealing member configurable to seal the at least one top portion filling member in the fluid-tight manner.

2. The apparatus of claim 1, wherein the sole portion includes the fluid-tight material and a sole portion hollow configurable to be filled with the liquid.

3. The apparatus of claim 2, further comprising:
at least one sole portion filling member attached to the sole portion in the fluid-tight manner and connecting with the sole portion hollow, the sole portion filling member being configurable to fill and empty the sole portion with the liquid; and
at least one sole portion sealing member configurable to seal the at least one sole portion filling member in the fluid-tight manner.

4. The apparatus of claim 1, wherein the top portion is removeably attached to the sole portion using one or more of a clasp, Velcro, a zip fastening, and at least one fastening belt.

5. The apparatus of claim 4, wherein one or more of the top portion and the sole portion are configured to be heated, cooled, or frozen.

6. The apparatus of claim 1, wherein the sole portion and the top portion are configured to receive ice cubes having sides of about 1.25 inches.

7. The apparatus of claim 1, wherein the at least one top portion filling member has an opening to receive the ice cubes having sides of 1.25 inches in length.

8. The apparatus of claim 1, wherein the fluid-tight material includes one or more of natural rubber, flexible plastic, polyvinyl chloride (PVC), and silicon.

9. The apparatus of claim 1, wherein the liquid includes one or more of water, a water solution, a polymer gel, an oil, and a wax.

10. The apparatus of claim 1, wherein the at least one top portion filling member is disposed on an upper edge of the top portion.

11. The apparatus of claim 1, wherein the top portion is secured around the foot using one or more of the following: a clasp, Velcro, a zip fastening, a tie-wrap, and a fastening belt.

12. The apparatus of claim 1, wherein the hollow is disposed around the foot and the ankle of the wearer.

13. The apparatus of claim 1, wherein the hollow is disposed around sides and a sole of the foot and around the ankle of the wearer.

14. The apparatus of claim 1, further comprising at least one insole for inserting in the top portion, the at least one insole being filled with the liquid and configured to be cooled, frozen, or heated.

15. The apparatus of claim 1, further comprising at least one insert for inserting in the top portion, wherein the top portion includes fixing means to fix the at least one insert in a desired position, the at least one insert being filled with the liquid and configured to be cooled, frozen, or heated.

16. An apparatus for providing thermal treatment to a foot of a wearer, the apparatus comprising:
a top portion having a form of a boot, the top portion including a fluid-tight material and configurable to be filled with a liquid; and
a gasket configured to secure the top portion around an ankle of the wearer and to prevent escaping of the liquid.

17. The apparatus of claim 16, wherein the gasket is secured using one or more of a clasp, Velcro, a zip fastening, and at least one fastening belt.

18. The apparatus of claim 16, wherein the liquid includes one or more of water, a water solution, a decoction, an infusion, an extract, and an oil.

19. A method for using an apparatus for providing thermal treatment to a foot and an ankle of a wearer, the method comprising:
disposing a top portion of the apparatus on the foot of the wearer;
filling the top portion with one or more of water, a water solution, one or more ice cubes, and an oil using a top portion filling member, the one or more of the water, the water solution, and the oil being preliminarily heated, cooled, or frozen;
sealing the top portion filling member in a fluid-tight manner using a top portion sealing member; and
optionally refilling the top portion with one or more of the water, the water solution, the one or more ice cubes, and the oil using the top portion filling member.

20. The method of claim 20, further comprising:
optionally inserting an insole in the top portion, the insole being preliminarily heated, cooled, or frozen; and
optionally attaching the sole portion to the top portion.
